(19) United States
(12) Patent Application Publication
(10) Pub. No.: US 2024/0215944 A1
(43) Pub. Date: Jul. 4, 2024

(54) Medical Follicles Assessment Device

(71) Applicant: PULSENMORE LTD., Omer (IL)

(72) Inventors: Elazar SONNENSCHEIN, Omer (IL); Elia PAZ, Mazkeret Batya (IL)

(73) Assignee: PULSENMORE LTD., Omer (IL)

(21) Appl. No.: 18/685,031

(22) PCT Filed: Aug. 11, 2022
(86) PCT No.: PCT/IL2022/050879

§ 371 Date: Feb. 20, 2024

(30) Foreign Application Priority Data
Aug. 23, 2021 (IL) 285798
May 18, 2022 (IL) 293114

(51) Int. Cl. A61B 8/08; A61B 8/00

(52) U.S. Cl. CPC A61B 8/0858; A61B 8/4455; A61B 8/462; A61B 8/466

(57) ABSTRACT

A device adapted to monitor the follicles and to assess the thickness of the endometrium of a subject comprises an elongated probe provided with an ultrasound array located at its distal end and a grip the axis of symmetry of which is at an angle with the axis of symmetry of the elongated probe.

### FIELD OF THE INVENTION

The present invention relates to the field of medical devices. In particular, the invention relates to devices and a method for monitoring follicles and assessing the thickness of the endometrium.

### BACKGROUND OF THE INVENTION

In vitro fertilization (IVF) is a method of assisted fertilization in which an egg is combined with sperm outside the body (“in vitro”). IVF can be performed by collecting the contents from a woman's fallopian tubes or uterus after natural ovulation or ovarian stimulation. In most IVF procedures, the ovaries are stimulated to make the follicles grow and produce mature eggs. Ovarian follicles play a major part in every IVF cycle; therefore, monitoring the development of the ovarian follicles is a critical part of the IVF process. Nowadays, IVF patients undergo several pelvic (vaginal) ultrasound scans performed by specialized personnel, i.e., nurses or physicians, during the natural menstrual cycle or the ovarian stimulation phase to confirm that the dosage of medication given to them to promote ovulation is correct and to determine when they are ready for egg collection.

In addition, the thickness of the endometrium changes during a person's menstrual cycle, but other factors can also prompt changes. It is therefore critical in many cases to measure the tissue thickness. The endometrium is the lining of the uterus. It is one of the few organs in the human body that changes in size every month throughout a person's fertile years. Each month, as part of the menstrual cycle, the body prepares the endometrium to host an embryo. Endometrial thickness increases and decreases during the process. Two hormones, estrogen and progesterone, prompt these cycles of endometrial growth, which is shed through menstruation if a pregnancy does not develop.

According to the Radiological Society of North America (RSNA), the endometrium is at its thinnest during menstruation, when it usually measures between 2-4 millimeters (mm) in thickness. The first half of the proliferative phase starts around day 6 to 14 of a person's cycle, or the time between the end of one menstrual cycle, when the bleeding stops, and before ovulation. At this phase, the endometrium begins to thicken and may measure between 5-7 mm. As the cycle progresses and moves towards ovulation, the endometrium grows thicker, up to about 11 mm. About 14 days into a person's cycle, hormones trigger the release of an egg. During this secretory phase, endometrial thickness is at its greatest and can reach 16 mm. Endometrial thickness is important in pregnancy. Healthcare experts link the best chances for a healthy, full-term pregnancy to an endometrium that is neither too thin nor too thick. This allows the embryo to implant successfully and receive the nutrition it needs. The endometrium gets thicker as the pregnancy progresses. Thus, monitoring and measuring the thickness is critical to a successful IVF and also for fertility preservation.

The monitoring includes assessing the number and size of the follicles on each ovary by ultrasound and by dedicated blood, saliva, or urine tests that measure the concentrations of relevant hormones. When the follicles are ready and are of the right size, around 18-20 mm, a trigger of hCG hormone injection is administrated. This trigger stimulates the follicles to discharge the mature eggs. Specialists then collect the mature eggs at a medical facility.

Because timing is essential in all IVF procedures, there is a need for daily monitoring of the ovarian follicles and the thickness of the endometrium, which creates a substantial burden on the patients and on the medical personnel and equipment. It is therefore clear that it would be highly desirable to be able to obviate the need for frequent visits to the medical facility where the assessment of follicles development is performed, thus reducing the burden and costs on the patient and the system alike.

It is a purpose of the present application to provide a device and a method, which achieve the aforesaid goal and substantially reduce the need to perform follicle monitoring and its assessment, as well as the thickness of the endometrium at a medical facility by specialized personnel.

It is another object of the invention to provide an affordable and accurate hand-held ultrasound imaging system suitable to be used for assessment purposes for IVF and Embryo Transfer (IVF-ET), as well as other fertility-related procedures. These may include, for instance, monitoring of spontaneous ovulation, natural preservative procedures by determining when there is no danger of pregnancy, and any other medical process requiring such monitoring.

It is a further purpose of the invention to provide a device and a method that reduce the load currently imposed on medical sites, thereby saving time and money for both patients and the health care system.

It is yet another object of the invention to provide a device that can be useful and easy to use for monitoring during various stages of the menstrual cycle, as well as an aid in other fertility procedures.

### SUMMARY OF THE INVENTION

The invention related to a device adapted to monitor the follicles and to assess the thickness of the endometrium of a subject, comprising an elongated probe provided with an ultrasound array located at its distal end and a grip the axis of symmetry of which (indicated by G-G in FIG. 14(a)) is at an angle with the axis of symmetry of said elongated probe (indicated by P-P in FIG. 14(a)).

Embodiments of the invention relate to a device that is adapted to be coupled to a hand-held device.

In the context of the present invention, the term “hand-held” should be given a broad meaning and may include any smart device, such as, for example, laptops which are not generally referred to as “hand-held.” In some embodiments, the device is configured to allow the subject to view content shown on its screen while operating the device itself. However, as will be further explained hereinafter, it is not necessary for the practical operation of the device that the operator be able to see the images generated by the ultrasound probe. Accordingly, the coupling to a hand-held device may be needed in some instances only for the purpose of facilitating communication between the device and an external target to which imaged and/or other data is to be transferred.

In one embodiment of the invention, the tip is detachable, and in another, the tip is disposable, e.g., a single-use or limited-use tip.

In some embodiments, the hand-held device is located in a cradle provided at the proximal end of the device. The electronics that operate the ultrasound array and the image acquisition and transferring elements are housed within the device's body, but some operations can also be performed using the processing power of the hand-held device. The hand-held device is in communication with the device's electronic component and receives data therefrom, which communication may be wired or wireless. In some embodiments, the data received by the hand-held device include ultrasound images. In other embodiments, the data received by the hand-held device include information indicative of the positioning of the tip relative to a desired location in the subject's body.

In some embodiments of the invention, the hand-held device is connected to the monitoring device via wire or wirelessly.

In some embodiments of the invention, the hand-held device is connected to a medical operator via internet or cellular communication.

An operator can guide the patient during the scan, or the patient can perform a self-scan procedure and upload the results (raw data or complete ultrasound image) to the cloud or transmit it to any other location where it can be stored or further reviewed or processed.

The resulting images can include 2D or 3D structures.

As will be appreciated by the skilled person, the physical properties of the device of the invention are also of importance. Too short a probe (as further described with reference to the drawings) may result in incomplete or low-quality images, while too long a probe may be conducive to inflicting harm by mishandling. Accordingly, the length of the probe shell, in one embodiment of the invention, is between 12 and 30 cm. Of course, devices of different configurations will have different probe shell lengths, depending on how the handle is structured, the connection of the shell to the cradle or the grip, as the case may be, etc. However, the abovementioned range is the typical working range for the device.

Similarly, the diameter of the probe should not be too big to avoid creating discomfort in the patient. Therefore, in one embodiment of the invention, the diameter of the probe, including its tip, should not exceed 30 mm.

The invention also encompasses a method for monitoring the size of the follicles and for assessing the thickness of the endometrium and/or the morphology of a subject, comprising instructing said subject to obtain specific ultrasound images using the device of the invention, based on the orientation of the probe.

In addition, it is possible to capture an image of the left or right side during a scan to illustrate to the patient how to search after a similar image.

This can be done in one embodiment by storing the baseline image, and when the patient performs a self-scan, the stored image is displayed. In another embodiment, when the patient scans, the system compares the baseline image to the current scan, and if a correlation is found, it provides an alert that prompts the patient to remain in the current location.

The method of the invention can be used for a variety of purposes, such as monitoring the follicles and/or assessing the thickness of the endometrium of a subject. Additional purposes include monitoring polycystic ovaries and the occurrence of natural ovulation.

The invention allows generating not only a 2D but also a 3D medical ultrasound image.

Scans can be performed in online or in offline modes, and in one embodiment, the method of the invention comprises performing an offline scan by the subject. Alternatively, it is possible to perform an online-guided scan guided by a healthcare professional.

The invention permits to perform assessments of the monitored parameter in a variety of ways. For instance, the assessment can be performed by AI, or by a human operator, or in a mixed-mode, in which the assessment is performed by providing an automated evaluation of the parameter of interest, followed by a verification of the value of said parameter by a human operator.

In one embodiment, the system provides suggestions to the subject for performing the scan during the scanning operation. In another embodiment, the system provides suggestions to a human operator or health professional for obtaining desired parameter values from ultrasound scans.

The invention also encompasses a system for monitoring the status of female reproductive organs in a subject, comprising:
a) a device comprising an elongated probe adapted to be coupled to a smart device, said probe being provided with a tip housing an ultrasound array;
b) instruction displayable to said subject to obtain specific ultrasound images using said smart device, based on the orientation of the probe;
c) at least one communication channel adapted to transmit the acquired ultrasound images, or data representative thereof, to a remote location.

In some embodiments, the device comprises electronic elements adapted to perform an ultrasound scan and electronic elements adapted to allow communication between said device and a smart device. In some cases, the electronic communication elements are adapted for wireless communication, and in other cases, the electronic communication elements are adapted for wired communication.

The system can utilize a variety of smart devices without limitation other than the ability of said smart device to perform the required communication with the device of the invention. In some instances, the smart device is selected from smartphones, tablets, and laptops.

There is no limitation to the way in which the system can provide operating instructions to the subject performing the scan. For instance, the instructions can be provided in a way selected from a printed form, displayed on a display, provided in audible form, or provided by a remote healthcare practitioner or technician via a communication channel. Additionally, the patient may self-scan without the aid of instructions based on previous training.

The communication channel between the subject and the remote healthcare practitioner or technician can be of any suitable type and can be, for instance, a video channel. Said video channel, in some embodiments, is established between the remote healthcare practitioner or technician and the smart device. In certain embodiments, the communication channel adapted to transmit the acquired ultrasound images, or data representative thereof, to a remote location is independent of the communication channel between the remote healthcare practitioner or technician and the subject.

All the above and other advantages of the invention will be better understood by reading the description of embodiments thereof, with reference to the appended drawings.

### BRIEF DESCRIPTION OF THE DRAWINGS

In the drawings:

FIG. 1 is a perspective view of an illustrative device according to one embodiment of the invention;

FIG. 2 is a front view of the device of FIG. 1;

FIG. 3 is a side view of the device of FIG. 1;

FIG. 4 is a perspective view of a device according to another embodiment of the invention, with a removable tip;

FIG. 5 is a perspective view of a device according to yet another embodiment of the invention, with a removable proximal section;

FIG. 6 shows a conventional probe for use in follicle development determination;

FIG. 7 shows the connection setup for the probe of FIG. 6;

FIG. 8 is an ultrasound image showing the ovaries;

FIG. 9 schematically shows the location of an array on the tip of a device, according to one embodiment of the invention;

FIG. 10(a-c) schematically shows a three-step procedure for imaging the ovaries, as explained below;

FIG. 11 illustrates the structure of a device according to another embodiment of the invention;

FIG. 12 illustrates the structure of a device according to yet another embodiment of the invention;

FIG. 13 is a cross-section of the device of FIG. 12, showing internal elements;

FIG. 14 (a and b) shows a device according to still a further embodiment of the invention, with a Wi-Fi module located near the top of the handle;

FIG. 15 (A-F) shows an example of a follicles assessment process according to a particular illustrative embodiment;

FIG. 16 illustrates a 3D imaging of follicles created by using three different cross-sections;

FIG. 17 illustrates the wireless pairing of a device of the invention with an external cradle;

FIG. 18 shows a wired connection of a device of the invention with an external cradle;

FIG. 19 shows a cradle devoid of physical pairing with the hand-held device it holds; and

FIG. 20 shows an alternative, cradle-less embodiment of the invention.

### DETAILED DESCRIPTION OF THE INVENTION

The device is adapted to identify follicle proprieties and the thickness of the endometrium. In one embodiment of the invention, the proximal end of the device is provided with a cradle that docks with the user's smartphone or the like smart device and allows a layman operator to produce ultrasound images without the need for a specialized ultrasound operator to be present in person, e.g., in the convenience of their home. In another embodiment of the invention, the device connects to portable devices such as smartphones, tablets, or any other suitable device via wireless or wired connections. Wireless connections may include, for example, Bluetooth or Wi-Fi, WIFI 5/6/7/8, UWB (ultra-wideband), or the like. In one embodiment of the invention, the follicles identification and the determination of the thickness of the endometrium are performed automatically using image processing and related processes, as further discussed below. In another embodiment of the invention, these determinations are carried out remotely by a specialist who inspects the images generated by the device. Images are transferred to the smart device coupled with the device of the invention via wire or wirelessly and then transmitted to the specialist for evaluation.

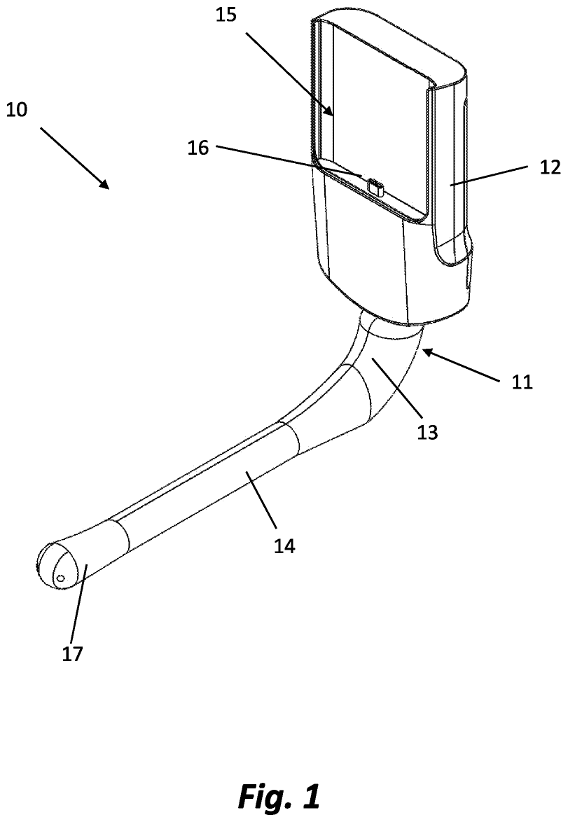

The device enables obtaining valid clinical data by instructing the patient to move the connected cradle in simple movements. If a cradle is dispensed with, as in the case of a wireless or wired connection, the same movements can be performed by handling the distal end of the device, e.g., that indicated at 13 in FIG. 1. Accordingly, without the need for any technical background, patients are able to scan and automatically send the images to the physician or other technician.

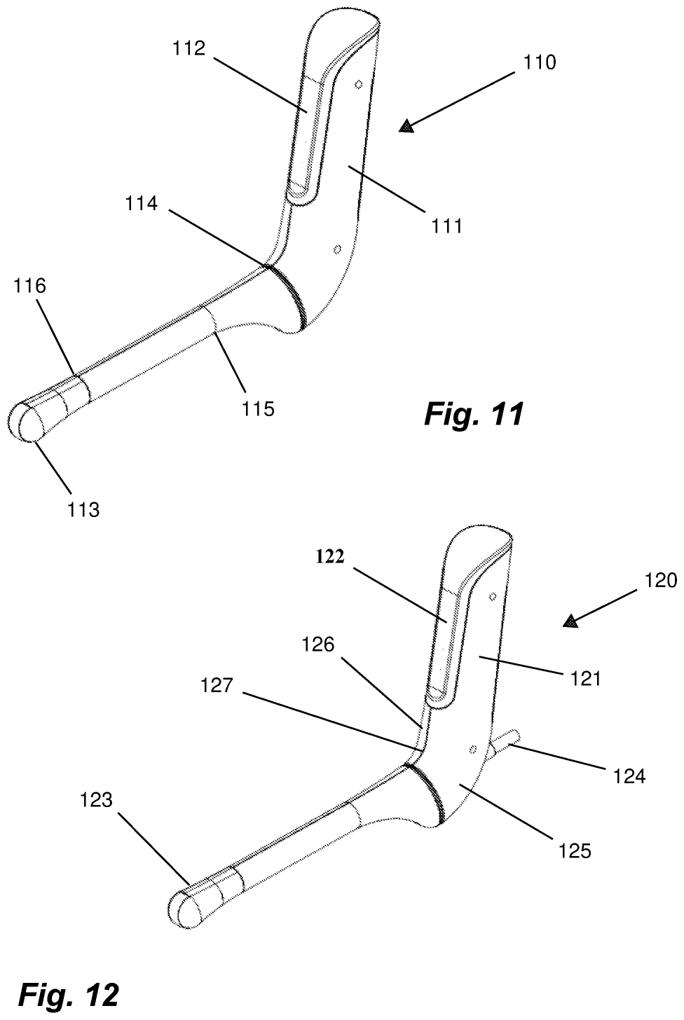

As will be understood by the skilled person, the device of the invention has a defined grip, which also defines the position of the ultrasound probe. For instance, when holding portion 13 of the device of FIG. 1, or handle 111 of the device of FIG. 11, the probe cannot freely rotate and, therefore, the direction in which the probe is scanning is defined, as opposed to prior art devices is which the elongated device may rotate and as such just by looking at the images acquired it is not possible to know whether scanning took place along a vertical, horizontal, or intermediate line.

As will be apparent to the skilled person, descriptions of operations provided with reference to 2D probes apply mutatis mutandis to 3D probes.

As used herein, the term “monitoring” refers to both visual inspection and parameter measurements, as the case may be. For instance, monitoring the development of follicles will, in many cases, involve measuring their size, as when IVF procedures are involved, but in some cases, it may be sufficient to ascertain qualitatively that follicles are developing, as may be, for example, in some cases when natural ovulation is to be confirmed, even though the measurement of follicles size may be desirable or necessary also when monitory natural ovulation. The same applies to the monitoring of the endometrium.

FIG. 1 is a perspective view of a device 10, according to one embodiment of the invention. It comprises a main body 11, which in this particular embodiment consists of a cradle 12 and a connecting section 13, adapted to connect to a disposable section, generally indicated by 14 (also referred to herein interchangeably as “probe shell”). In other embodiments, the connecting section 13 may also be disposable and not integral with cradle 12. Cradle 12 has a void part 15, adapted to receive a smartphone or other hand-held device, and a communication connector 16, which is adapted to fit with the communication port of the hand-held device. The tip, 17 (also referred to interchangeably herein as “probe head”), holds a convex transducer with an array of active elements and other electronics, which adjusts the connection to the mainboard and sensors. The width of the void part 15 may be expandable, e.g., by providing displaceable side walls (not shown) to accommodate diverse sizes of hand-held devices.

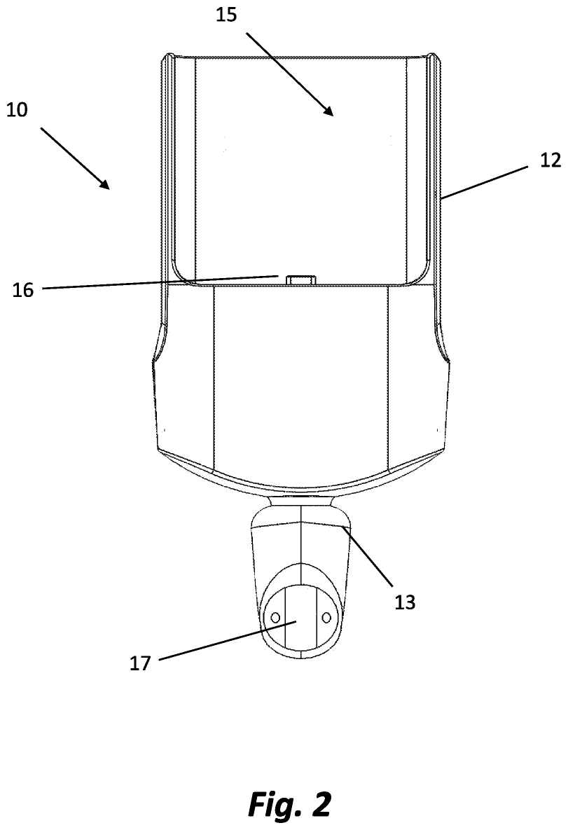

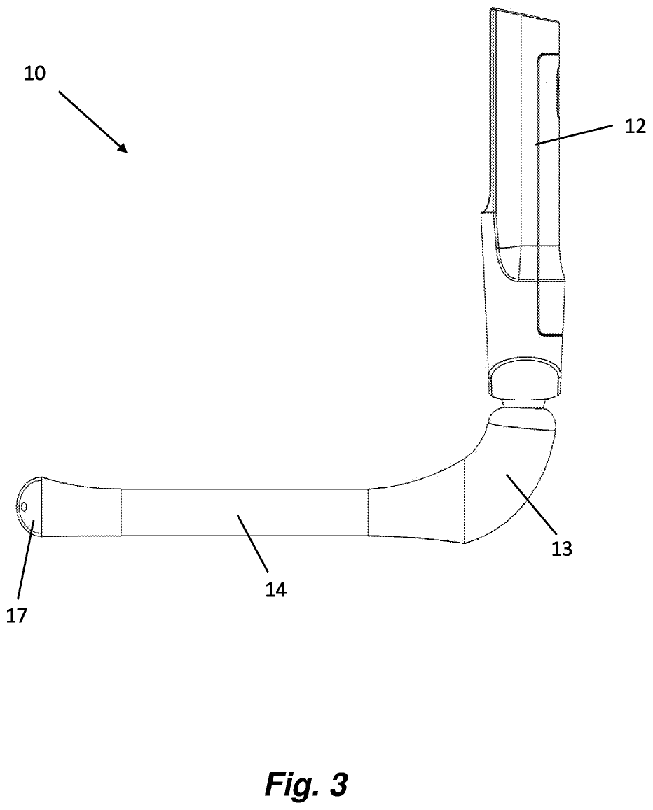

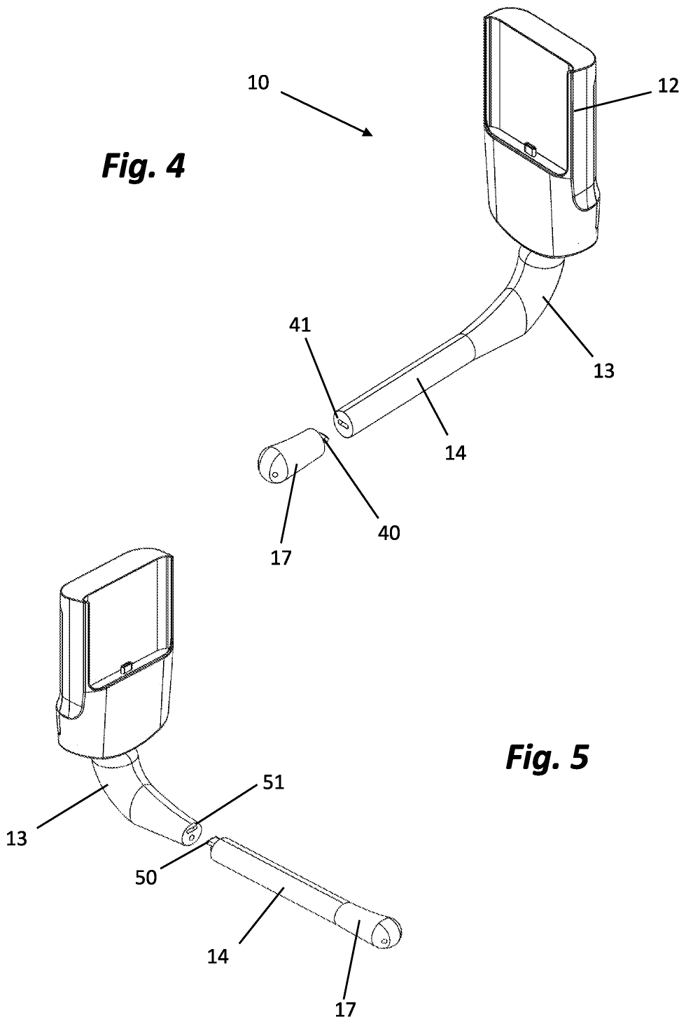

FIGS. 2 and 3 are front and side views, respectively, of the device of FIG. 1 and further illustrate it. FIG. 4 illustrates a device in which tip 17 is detachable and connector 40 and connecting port 41, which allow it to be in communication with the hand-held device housed in cradle 12 (not shown in the figure). In some embodiments, tip 17 may be disposable.

FIG. 5 illustrates a device according to another embodiment of the invention, in which portions 14 and 17 are detachable together. In this embodiment, connector 50 and port 51 allow communication with the hand-held device housed in cradle 12 (not shown in the figure).

The ultrasound transducer (probe) can be chosen from several forms, such as a linear array, phased array, matrix array, or any other 1D, 1.5D, 2D, 2.5D, and 3D array, suitable for detecting and identifying follicles and the thickness of the endometrium. The transducer can have 1 to 512 elements made of piezoelectric material, Micro Electro Mechanical System (such as CMUT), or a combination of both (such as PMUT) with central frequency from 2 MHz to 20 MHz, i.e., a 64, 128, 192, 256 or 512 standard piezo or bulk piezo ceramic elements or CMUT/PMUT silicon-based probe. On the transvaginal probe, there are also sensors in several positions, like on the tip of the head of the transducer, on the probe shell, and inside the head of the probe, to measure the intensity of the contact between the probe (transducer) and the tissue for safety, and also to identify excessive pressure and warn the user thereof to prevent injury.

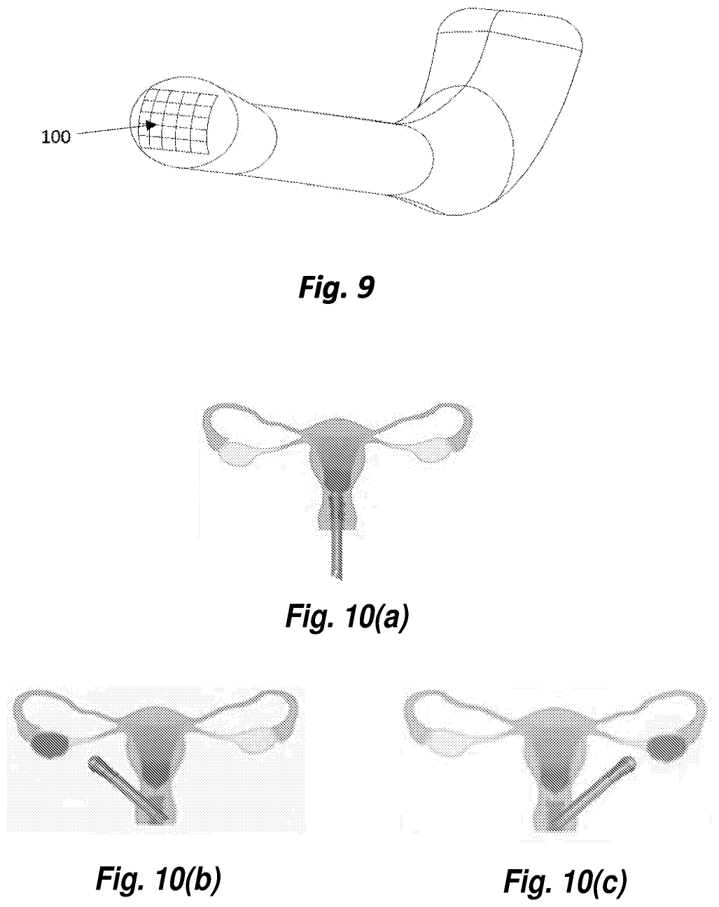

In some embodiments, the transvaginal probe has the ability to steer for a larger aperture, with mechanical or electronic aid, which expands the range of the scan axis or adds another scan axis to acquire a better ultrasound image and is adapted to generate a 3D medical ultrasound image, when the option is given to scan in two axes relative to the body being examined. Such embodiment is implemented with a 2 to 10 mm diameter miniature electrical motor, piezo motor, or cable connected to the rotating probe. When the motor is located in the cradle, illustrative cable dimensions are 0.1 mm to 5 mm. Suitable gear is also implemented as required. In addition, the probe can be disconnected from the device and replaced as a result of wear and tear, or, as explained above, it can be disposable after use. The transvaginal probe may further be fitted with a dedicated disposable or reusable cover to achieve better acoustic coupling and maintain sterility. Another option for the ability to steer is by electronic means with beamforming and/or phased array methods, with an array in the form of a two-dimensional matrix or two linear arrays set at some angle between them (15°-90°), as schematically shown at 100 in FIG. 9.

In another embodiment of the probe, the transducer tip 17 itself can be single-use, and part 14, which connects the transducer to the cradle can be self-propelled with a plastic sleeve. In this case, self-propulsion is achieved using air or a water pump. The pump, in some embodiments, is a miniature device with a size of 2 to 20 mm and is implemented in the cradle (not shown).

The cradle is further provided with a mechanism adapted to adjust the device's ergometry for optimal usability by the patient. Various ways to do so are clear to the skilled person and are not illustrated for the sake of brevity. The cradle also contains an electronic board with hardware to support the operation of the device and to interface with a mobile phone.

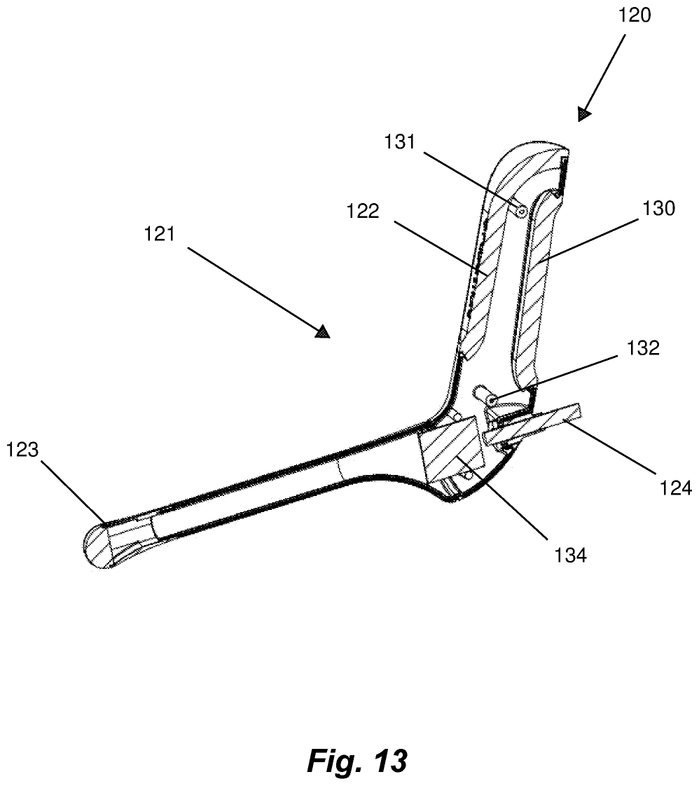

FIG. 11 and FIG. 12 illustrate two alternative embodiments. Both embodiments operate without a built-in screen or with an associated cradle for a hand-held device, like that described with reference to FIG. 1. The difference between these two embodiments is in the communication interface. While device 110 of FIG. 11 communicates wirelessly, e.g., via Wi-Fi or Bluetooth, device 120 of FIG. 12 communicates via wired connection 123 (shown as a truncated line in the figure). Other than for this difference, the two embodiments of FIGS. 11 and 12 are identical and, therefore, additional details are described in FIG. 13 with reference to the embodiment of FIG. 12, which also apply to the device of FIG. 11.

As will be apparent to the skilled person, when a device is used according to an embodiment in which all electronic elements needed for performing the ultrasound scan and those needed for communicating, whether wirelessly or wired, with an external smart device are located inside the device itself, the subject can easily move the probe with one hand, while performing the scan, while holding the smart device (for instance, a smartphone, a tablet, a laptop, etc.) with the other. This allows the subject to view the information relevant at the time on the smart device's screen.

However, in many cases, there is no need for the user to watch the screen of an associated device showing images. Because the movements the user has to perform are well defined, as will be further explained with reference to FIG. 10, and images are acquired when performing them. It is not generally expected of the user to change the scanning protocol as a result of an image she sees on the screen. In some cases, if the user is experienced at performing the scanning of the ovaries or if an expert is instructing the user to perform specific scans, the images shown on the screen of an associated device may be useful. Moreover, the ability to see what is being imaged may be important to some users to reassure them that the device is functioning.

The device's handle (grip) can include all necessary electronics by means of an Analog Front End and microcontroller (or an FPGA can replace this element). It is also possible to relocate all electronics to a cradle or close to the display (in the case of a Tablet/computer). An AFE can be implemented from one or two chipsets of 8/16/32/64 channels that include transmitters, receivers, Low Noise Amplifier (LNA), ADC, and or processors such as micro-controller/FPGA.

Returning to FIGS. 11 and 12, other common elements are shown. In one embodiment, grips 112 and 121 are provided with actuation switches 112 and 122, which operate the ultrasound probe located in tips 113 and 123. Since handles or grips 111 and 121 are held in the patient's hand during the operation of the ultrasound device, a simple application of pressure or release thereof turns the probe on or off, as the case may be. An additional or alternative pressure switch can be located at the backside of the grip (not shown in FIGS. 11 and 12 and illustrated in FIG. 13). Suitable switches and switch assemblies adapted for this purpose are well known in the art and therefore are not discussed herein for the sake of brevity.

The structure of the device of the invention can be, but not necessarily is, monolithic. For instance, in some embodiments, the device can be made of separate parts adapted to be assembled, some of which may be detachable. For instance, with reference to FIG. 11, numerals 114, 115, and 116 may each represent an assembly/disassembly line, where the device can be taken apart for maintenance or parts replacement.

Looking now at FIG. 13, which is a cross-section of a device of FIG. 12, the backside of grip 121 is shown, with surface 130, which may function as an additional or alternative actuation switch or simply be a static part of the grip 121. Numerals 131 and 132 indicate a cross-section of supporting fasteners adapted to keep two parts of the device's outer surface together when parts 125 and 126 of FIG. 12 are joined along line 127. In the particular embodiment of FIG. 13, electronic elements, such as ultrasound probe controller, communication module, etc., are housed in part 134, which cooperates with both probe 123 and communication line 124.

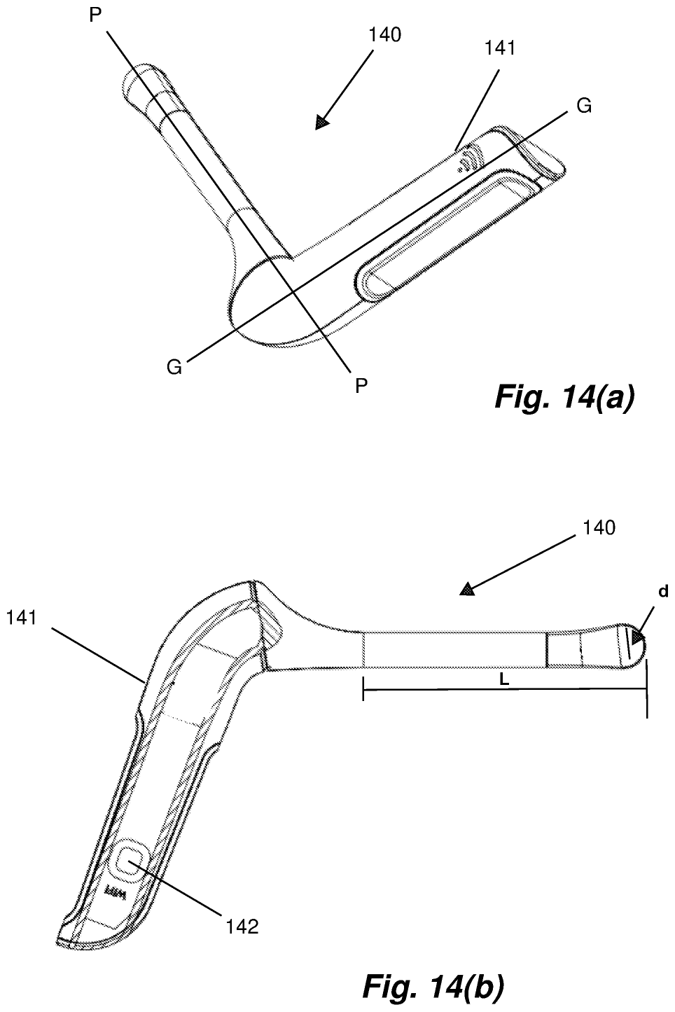

In cases when the wired communication link is replaced by wireless connections, the wireless modules, such as Wi-Fi, Bluetooth, etc., are also located in part 134. In other embodiments, of course, electronics can be distributed inside the body of device 120 at more than one location, as expedient in each particular case. This is illustrated in FIGS. 14 (a) and (b), which show an embodiment using a Wi-Fi wireless module for communication. In this case, the Wi-Fi element 142 is located inside grip 141 and towards its upper end.

In some embodiments, the device of the invention can be adapted to communicate with a remote device, e.g., via a home router or a hot spot, and transmit the information and images collected by the ultrasound probe to a remote location.

FIG. 14 also shows the length L of the probe shell and the diameter d of the probe head, discussed above.

Embodiments of the invention also include more than one communication channel, typically but not limitatively, two communication channels. The first communication channel connects the ultrasound device to a smart device, e.g., a smartphone, a tablet, or another hand-held device. The second communication channel connects the device or a smart device connected to it to a router (or cellular communication).

Image processing can be performed differently in various embodiments of the invention, such as locally, on a computer or other mobile or smart devices, or raw image data can be sent to the cloud and processed in the cloud or by a remote smart device.

Regardless of the physical structure of various devices according to alternative embodiments of the invention, the ultrasound image is analyzed with the help of a suitable application using image processing methods known per se in the art, which run on a mobile device, tablet, or PC, to determine the patient's follicle status and the thickness of the endometrium. The acquisition of the ultrasound images can be made with the help of an application that instructs the user on how to perform the scan, or by providing a visual or written illustration of the scanning steps, or by direct instruction by a trainer. Instead of analyzing the images in the cradle or in the hand-held device, in one embodiment of the invention, the ultrasound images are delivered to a remote location and analyzed, e.g., using an image processing tool in cloud computing and/or by a medical specialist. As a result, the device permits to generate an indication of the IVF cycle status and to give recommendations on how to proceed with the process.

The determination of the thickness of the endometrium using transvaginal ultrasound is well known in the art, and its importance has been discussed [Gupta A, Desai A, Bhatt S. Imaging of the Endometrium: Physiologic Changes and Diseases: Women's Imaging. (2017) Radiographics: a review publication of the Radiological Society of North America, Inc. 37 (7): 2206-2207]. Accordingly, no discussion of this measurement is needed and suffices it to say that the ultrasound images of the endometrium obtained using the device of the invention can be treated as described herein with reference to the follicles, but automatic software evaluation and/or by qualified healthcare personnel.

The cradle, in some embodiments, may also use information from other internal or external sensors, such as accelerometers, altitude, clock, magnetic field, pressure, temperature, and gyroscope, to generate a precise location of the cradle that is displayed on the mobile phone and the video sound that is sent to the remote location. Using the information so acquired, the medical specialist can better understand the configuration of the probe while scanning.

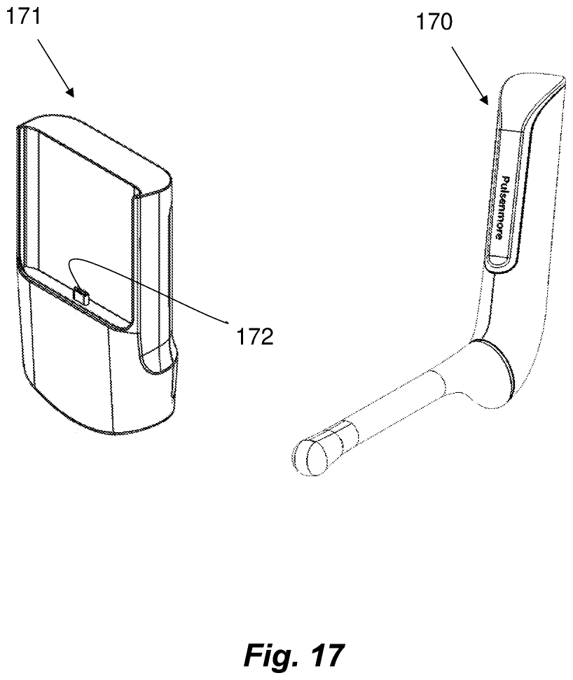

FIG. 17 shows an arrangement according to one embodiment of the invention in which the ultrasound device 170 is paired with cradle 171. While device 170 is handled with one hand, cradle 171 may be handled with the other or, depending on the user's preference, may be set aside and not looked at during the scan. In this embodiment, a wireless connection is established between device 170 and a corresponding wireless element in cradle 171. A smart device such as a smartphone (not shown) is placed in the cradle and is in connection with it via connector 172. Accordingly, images and data transmitted to cradle 171 by device 170 are received in the smart device, which may then transmit them to a remote location via an independent wireless connection.

Alternatively, cradle 171 may also be connected to a router to which the smart device is also connected (or, alternatively, the smart device can use a cellular Internet connection), so data can be transmitted to a remote location by both or either of the smart device and cradle 171.

In some embodiments, the communication between device 170 and cradle 171 is also performed via a router.

It should be understood that the invention may operate differently from other devices that transfer images and are known in the art insofar as the system of the invention may employ two open communication channels simultaneously when, for example, the cradle 171 may function as a router or the smart device can operate as a hotspot during the data transfer, which may simultaneously take place, in one embodiment, between the device 170 and cradle 171 and between the smart device and a remote location.

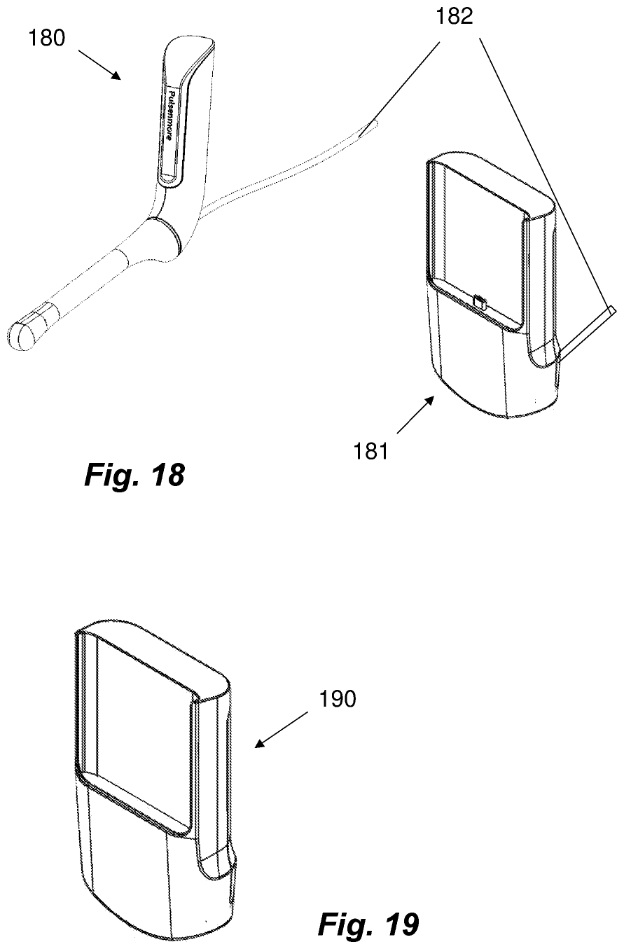

The embodiment of FIG. 18 is similar to that of FIG. 17, but the connection between device 180 and cradle 181 is performed via wire 182.

FIG. 19 shows an alternative cradle 190. This cradle does not have a connector such as 172 of FIG. 17, and it only functions to hold the smart device without a direct connection to the cradle. In this embodiment, the ultrasound device (e.g., 170 of FIG. 1) transmits images and data to cradle 190, either wirelessly or via a wired connection, and then a separate wireless connection is established to transfer said images and data to the smart device in the cradle, which may then send them to a remote location. Cradle 190 may be equipped with two independent wireless elements, one for connecting to the ultrasound device and the other for connecting to the smart device, or may have a single wireless element that will disconnect from the ultrasound device when its operation is completed and connect to the smart device in the cradle. Alternatively, the wireless component of the cradle may hop between the two different connections during the operation of the ultrasound device.

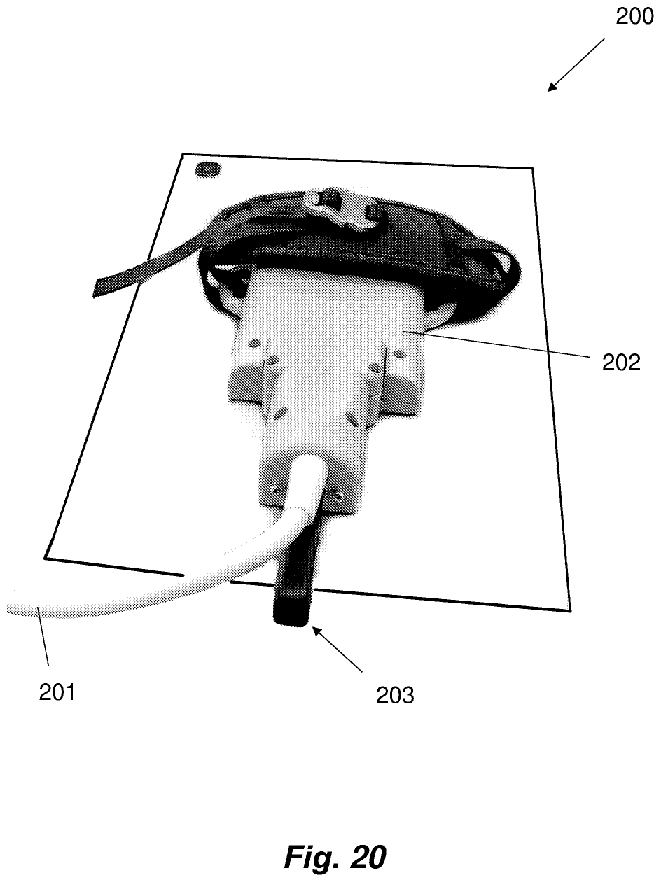

FIG. 20 shows an alternative, cradle-less embodiment of the invention. In this embodiment, a smart device 200 (in this illustration, a tablet) is coupled to an ultrasound device such as 180 of FIG. 18, via cable 201. The electronic data handling apparatus needed to receive the data sent by device 180 and format it such that it is receivable by tablet 200 is located in housing 202, which is mechanically coupled to tablet 200. A connector, 203, connects the output of data handling apparatus 202 with the input of tablet 200. Both the output of data handling apparatus 202 and the input of tablet 200 are not seen in the figure since they are covered by connector 203, which is plugged in.

### Example of Use

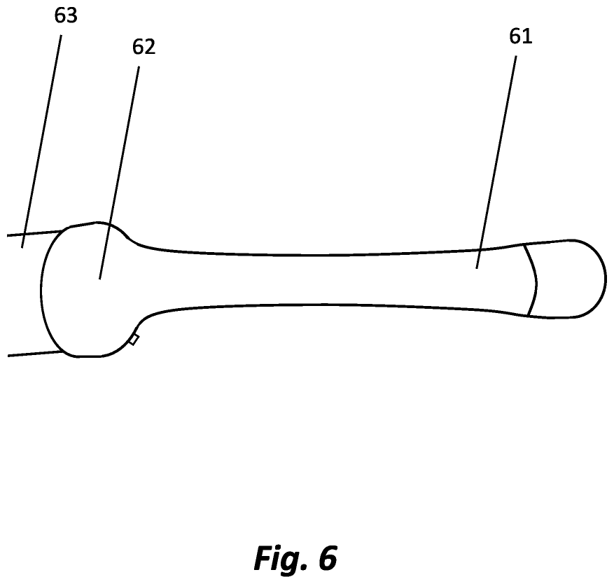

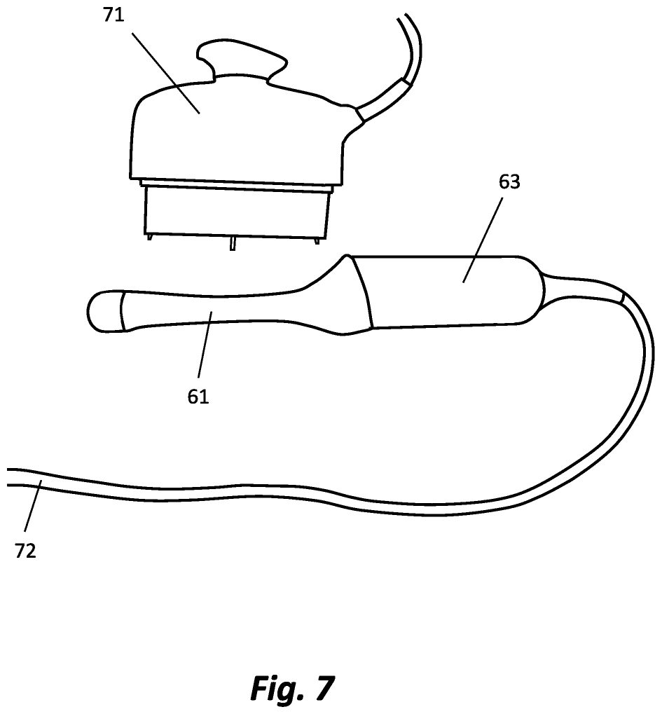

The following example will illustrate the use of the device of the invention through an actual example of use. An examination is performed as customary for the conventional probe (shown in FIGS. 6 and 7. Such conventional probes, such as that indicated by numeral 61, are attached to external equipment via connector 71 (FIG. 7), which is connected to it through cable 72.

Moreover, a transducer marker 62 is provided on handle 63 to ensure the proper orientation of the probe by the user. This need is obviated by the particular configuration of the device of the invention.

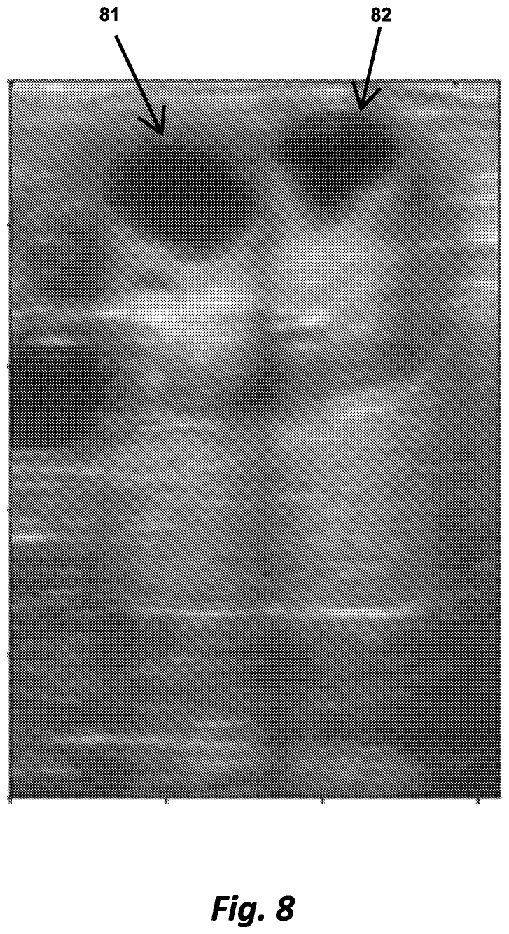

Using the device of the invention, the ovaries can be seen as illustrated in FIG. 8, in which the two ovaries are indicated by numerals 81 and 82. An illustrative example of self-scan protocol adapted for use by an unskilled individual, which is suitable for use with the device of the invention, is the stepwise standardized approach to monitoring the follicles and the thickness of the endometrium, described by Abuhamad, Alfred, et al. (Ultrasound in obstetrics and gynecology: a practical approach, 2014). This protocol is applied in three simple steps: 1) The user holds the transducer straight in to take the first image of the uterus FIG. 10 (a); 2) The user moves the transducer to the left to image the left ovary FIG. 10 (b); 3) The user moves the transducer to the right to image the right ovary FIG. 10 (c). In FIG. 10 (a), when imaging the endometrium, measurements are taken in the long axis or sagittal plane, ideally on transvaginal scanning, with the entirety of the endometrial lining through to the endocervical canal in view.

The invention utilizes image processing, machine learning, and artificial intelligence to identify and measure the follicles and the thickness of the endometrium seen in the image acquired with the device's assistance. These steps include:
1) Preprocessing the ultrasound image to obtain a simpler image with the same intensity range; 2) Using advanced segmentation algorithms to detect suspicious regions of interest; 3) Extracting shape and texture properties for each segment, using machine learning and AI methods to identify follicles and the thickness of the endometrium contour from image features; 4) By using machine learning and AI methods, selecting the best image or images to measure the sizes of the follicles and endometrium from all the frames obtained; 5) Fitting a known contour (such as ellipse or rectangle) to the detected area; 6) Calculating the follicles dimensions and the thickness of the endometrium (if fit to an ellipse, minor and major axis dimensions, and if to a rectangle, the thickness). For instance, identifying follicles can be performed by an approximation to an ellipse shape, and either or both 2D or 3D imaging can be used with a suitable 3D probe. In the case of 2D imaging, an ellipse is approximated, and in the case of 3D imaging, an ellipsoid. The two axes of the ellipse, or three axes in the case of an ellipsoid, are used to determine the size of the follicle based on a 2D or a 3D model. With respect to the endometrium, it should be measured in the long axis or sagittal plane, ideally on transvaginal scanning, with the entirety of the endometrial lining through to the endocervical canal in view.

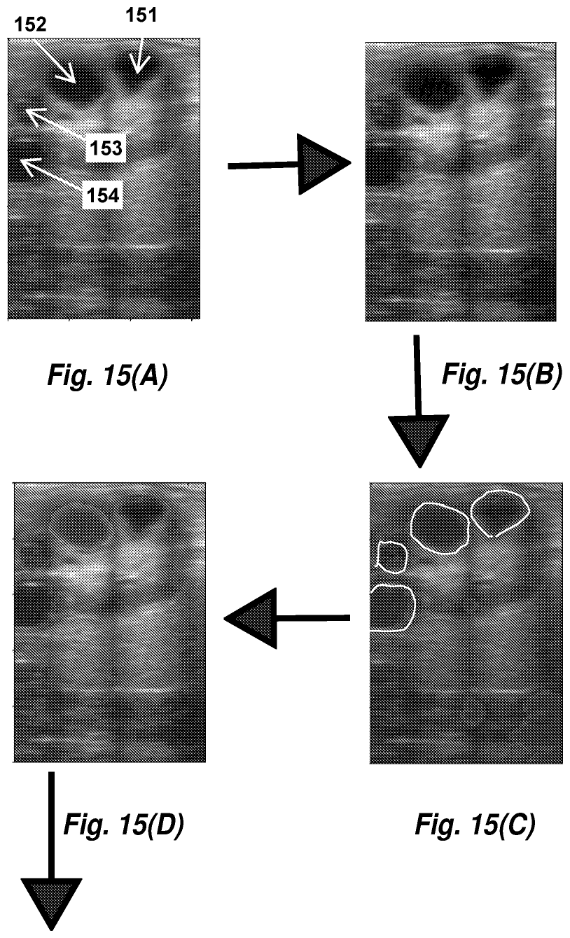

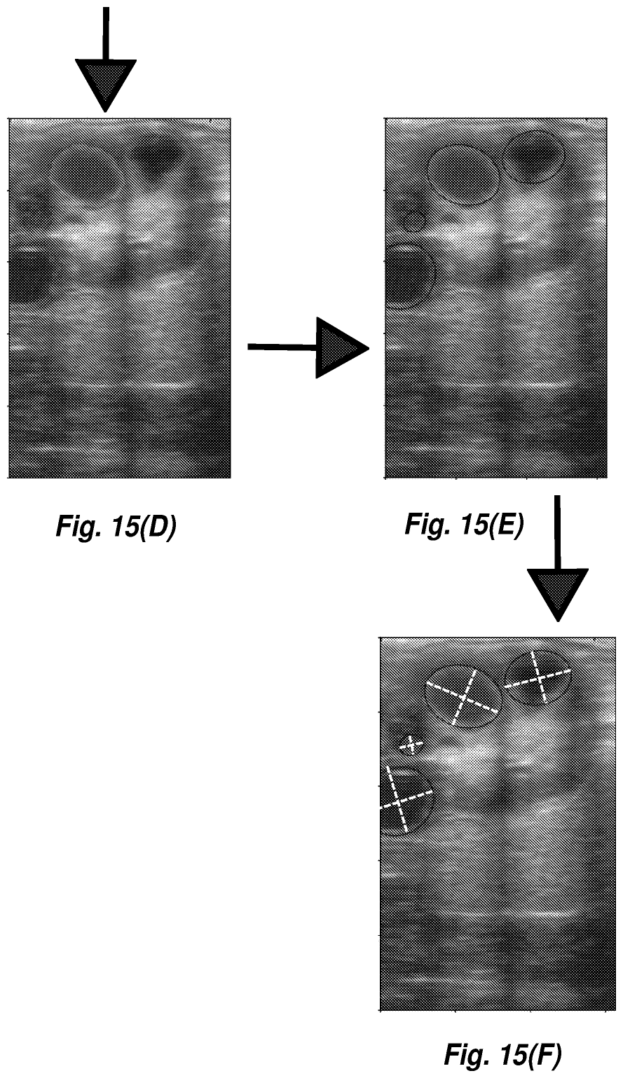

FIG. 15 shows one illustrative procedure for estimating the number and size of follicles. Image processing methods that are well known in the art per se are not described in detail for the sake of brevity. However, persons skilled in the art of machine learning and artificial intelligence (AI) will readily appreciate the use made of such methods in the context of the invention and will be able to implement such methods without the need for a lengthy discussion.

FIG. 15A shows an area in which the presence of follicles has been found in the ultrasound scan image. These appear as darker spots, identified as 151-154 in the figure. In this example, the ultrasound image is first preprocessed to obtain a simpler image with the same intensity range (FIG. 15B). A segmentation algorithm (known per se in the art) is then used to identify regions suspected of being of interest. These are identified in FIG. 15C by the white circles added for illustration. In practice, these can be identified by the segmentation software in any suitable way, e.g., by coloring the suspected areas in a different color. The identified areas are then processed using machine learning/AI methods known per se to extract their shape and texture and obtain follicles contours (FIG. 15D).

From the contours obtained in FIG. 15D, ellipses are fitted to the various detected follicles to fit their contour, as shown in FIG. 15E. Finally, the follicles' minor and major axis dimensions are calculated (FIG. 15F), and a report detailing follicle number and their dimensions is generated.

As will be apparent to the skilled person, although each image processing step is per se known and can be performed by skilled persons, the sequence of steps as applied to the invention is novel and provides significant unexpected advantages.

It should be understood that the invention allows for great flexibility in performing the desired operation. For instance, while in some cases, the system and the healthcare practitioner in charge of the patient will rely on the determinations made by the AI system, in other cases, there may be a preference for the performance of the evaluation, e.g., of the size of the follicles, by a human operator. The invention allows for both options and combinations thereof, in which the system proposes data determined by AI, and said data is manually verified by an operator.

All the above image processing and manipulation stages are known, per se in the art, and, therefore, they are not described herein in detail. It is their combination, in the context of the present invention, that leads to the valuable output obtainable using the device of the invention.

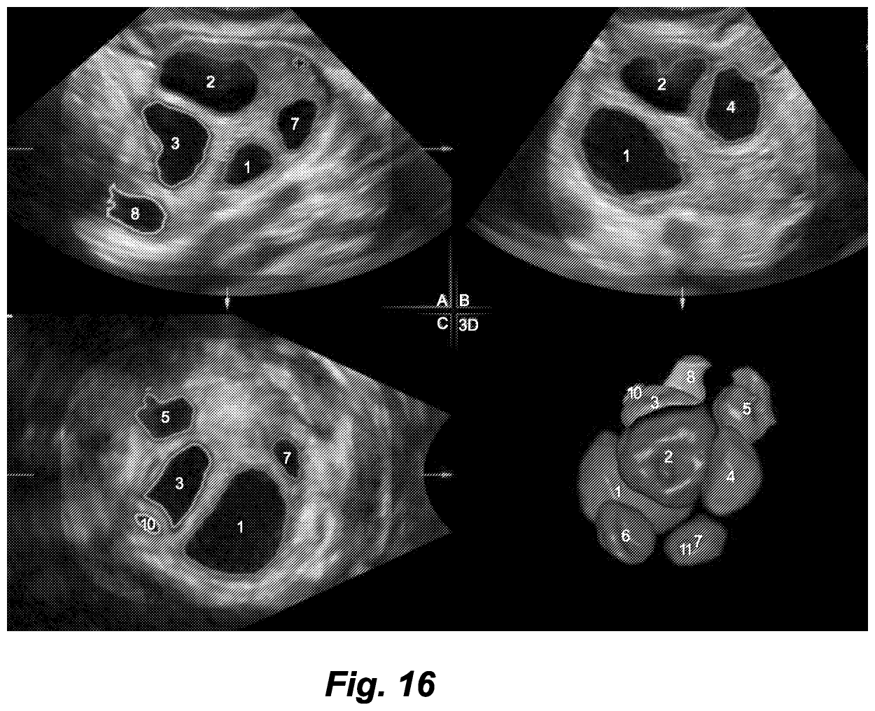

FIG. 16 shows the result of a 3D imaging of the follicles. Three different cross-sections, A-B-C, show different follicles (indicated by numerals 1-11) and allow for the creation of a 3D image, shown in the right-hand bottom quadrant of the figure.

As explained, the invention allows a patient to perform a self-scan without the help of a health professional, such as an ultrasound technician or a physician. However, in some cases, it is advantageous to perform the scan in cooperation with the patient's physician or other health professional. The following is an example of such a cooperative activity, it being understood that this example is only meant to illustrate the ability afforded by the invention. The skilled person will easily understand that many other different scenarios will apply.

The following flow exemplifies the above:

### Set Appointment

The physician receives a text message or an email with the appointment containing a link to the ultrasound meeting (generated with the patient's unique key).

At the appointed time, the physician initiates the video call with the patient. Then, he clicks the link to open a web application associated with the device. He accesses the web application using the appropriate credentials (username, password, etc.) and clicks the start button to start a video session with the patient.

### Starting the Ultrasound Scan

The physician can remotely start and control the ultrasound scan, record video clips, snapshots, etc. In one alternative, the Physician can decide if the patient sees the ultrasound scan in real-time or not.

### Post Scan

All the video recorded during the scan is uploaded to the cloud or a remote server, as the case may be, and becomes available for the physician to view offline and document in the appropriate system.

As will be readily appreciated by persons skilled in the art, the present invention excels in usability and generality over prior art devices. For instance, the device described in U.S. patent application Ser. No. 16/802,344 relates to a device and method for self-home scanning to monitor follicle size. The described method presents the substantial disadvantage that a specialist who operates the device must perform an initial scan. In the same initial scan, the specialist marks the location of the physiological elements in the subject's ovarian area with the help of permanent markers. This is contrary to the teachings of the present invention, which do not require an expert scan to mark anatomical structures and do not use physical markers at all. Moreover, the device of U.S. Ser. No. 16/802,344 becomes specific to each user after the markers are in place, while the device of the invention is not specific to any user after initial use.

1. A device adapted to self-monitor follicles and to assess a thickness of an endometrium of a subject, the device comprising a grip and an elongated probe, the elongated probe provided with an ultrasound array located at its distal end;
the grip angularly configured for self-handling by a user during a self-monitoring activity;
the grip and elongated probe each having an axis of symmetry, the axis of symmetry of the grip being at an angle with respect to the axis of symmetry of said elongated probe.

2. The device of claim 1, which is adapted to be coupled to a hand-held device.

3. The device of claim 1 further comprising a probe tip, wherein the probe tip is selected from detachable, disposable, or single use.

4. The device of claim 1, wherein electronics that operate the ultrasound array and image acquisition and transferring elements are housed within a body of the device.

5. The device of claim 1, which is adapted to transmit a raw ultrasound image or a signal representative thereof to a hand-held device that includes electronic processing components and a display.

6. The device of claim 5, wherein data received by the hand-held device include information indicative of a positioning of a probe tip relative to a desired location in the subject's body.

7. The device of claim 1, wherein the hand-held device is connected to a monitoring device via wire or wirelessly.

8. The device of claim 1, wherein a length of a probe shell is between 12 and 30 cm.

9. The device of claim 1 further comprising a probe tip, the probe tip comprising a diameter, wherein the diameter of the probe tip is up to 30 mm.

10. A method for self-monitoring follicles and to assess a thickness of an endometrium of a subject, the method comprising allowing a subject to, using a device comprising a grip and an elongated probe, self-scan to obtain specific ultrasound images based on an orientation of the elongated probe;
the elongated probe provided with an ultrasound array located at its distal end;
the grip angularly configured for self-handling by a user during a self-monitoring activity;
the grip and elongated probe each having an axis of symmetry, the axis of symmetry of the grip being at an angle with respect to the axis of symmetry of said elongated probe.

11. The method of claim 10, comprising monitoring natural ovulation.

12. The method of claim 10, comprising performing an offline scan by the subject.

13. The method of claim 10, comprising performing a healthcare professional online guided scan.

14. A system for self-monitoring follicles and to assess a thickness of an endometrium of a subject, the system comprising:
a) a device comprising an elongated probe and a grip, the elongated probe provided with an ultrasound array located at its distal end and the grip angularly configured for self-handling by a user during a self-monitoring activity, the grip and elongated probe each having an axis of symmetry, the axis of symmetry of the grip being at an angle with respect to the axis of symmetry of the elongated probe;
b) instruction displayable to said subject to obtain specific ultrasound images using a smart device, based on an orientation of the elongated probe;
c) at least one communication channel adapted to transmit the acquired ultrasound images, or data representative thereof, to a remote location.

15. The system of claim 14, wherein the device comprises electronic elements adapted to perform an ultrasound scan, and electronic elements adapted to allow communication between said device and the smart device.

16. The system of claim 15, wherein the smart device is selected from smartphones, tablets, and laptops.

17. The system of claim 15, wherein the instructions are selected from printed, displayed, or audible instructions.

18. The system of claim 15, wherein the instructions are provided by a remote healthcare practitioner or technician via a communication channel.

19. The system of claim 15, wherein the communication channel adapted to transmit the acquired ultrasound images, or data representative thereof, to a remote location, is independent of the communication channel between the remote healthcare practitioner or technician and the subject.

20. The system of claim 15, adapted to:
(a) store one or more baseline image(s) of areas of interest;
(b) when a patient performs a self-scan, display the stored image to assist the patient in locating and scanning a correct image.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)
